(19) United States
(12) Patent Application Publication
(10) Pub. No.: US 2020/0224169 A1
(43) Pub. Date: Jul. 16, 2020

(54) PREPARATION METHOD OF MESENCHYMAL STEM CELL-DERIVED EXOSOMES BASED ON DRUG PRETREATMENT

(71) Applicant: Fuwai Hospital, Chinese Academy of Medical Sciences and Peking Union Medical College, Beijing City (CN)

(72) Inventors: Yuejin Yang, Beijing City (CN); Peisen Huang, Beijing City (CN); Guihao Chen, Beijing City (CN)

(21) Appl. No.: 16/833,405

(22) Filed: Mar. 27, 2020

Related U.S. Application Data
(63) Continuation-in-part of application No. PCT/CN2018/091843, filed on Jun. 19, 2018.

(51) Int. Cl. C12N 5/0775; A61K 35/28; A61P 9/10

(52) U.S. Cl. CPC C12N 5/0663; A61P 9/10; A61K 35/28; C12N 5/0667

(57) ABSTRACT

A method for preparing mesenchymal stem cell-derived exosomes on the basis of drug pretreatment, the method for preparing mesenchymal stem cell-derived exosomes comprising: using a statin to pretreat mesenchymal stem cells, and culturing the treated mesenchymal stem cells to collect exosomes secreted thereby. Also provided is an application of a statin in preparing a preparation for promoting the anti-apoptotic abilities and/or homing abilities of mesenchymal stem cells; and further provided is an application of a statin in preparing a preparation for promoting mesenchymal stem cells to secrete exosomes having myocardial infarction microenvironment-improving effects and/or myocardial repair abilities.

### CROSS-REFERENCE TO RELATED APPLICATIONS

This application is a continuation-in-part of International Application No. PCT/CN2018/091843, filed on Jun. 19, 2018, which is hereby incorporated by reference in its entirety.

### TECHNICAL FIELD

The invention relates to a preparation method of mesenchymal stem cell-derived exosomes based on drug pretreatment, specifically, an efficient preparation method of mesenchymal stem cell-derived exosomes based on statins pretreatment.

### BACKGROUND

WHO statistics show that cardiovascular diseases were the leading cause of death in the world in 2016 (about 17.6 million, 32.2%), among which 9.48 million died of ischemic heart disease (17.3%) (Collaborators GM. Global, regional, and national under-5 mortality, adult mortality, age-specific mortality, and life expectancy, 1970-2016: a systematic analysis for the Global Burden of Disease Study 2016. The Lancet 2017; 390(10100):1084-1150; Collaborators GCOD. Global, regional, and national age-sex specific mortality for 264 causes of death, 1980-2016: a systematic analysis for the Global Burden of Disease Study 2016. The Lancet 2017; 390(10100):1151-1210). Acute myocardial infarction (AMI) causes massive myocardial ischemic necrosis, and the replacement by scar tissues in turn leads to heart failure and death. Current therapeutic approaches cannot effectively regenerate and repair myocardium. In recent years, high expectation has been given to stem cell transplantation, especially mesenchymal stem cell (MSCs) transplantation such as bone marrow-mesenchymal stem cells (BM-MSCs) transplantation, in the treatment of AMI (Orlic D, Kajstura J, Chimenti S, Jakoniuk I, Anderson S M, Li B, Pickel J, McKay R, Nadal-Ginard B, Bodine D M and others. Bone marrow cells regenerate infarcted myocardium. Nature 2001; 410(6829):701-705; Fisher S A, Doree C, Mathur A, Martin-Rendon E. Meta-Analysis of Cell Therapy Trials for Patients With Heart Failure. Circulation Research 2015; 116(8):1361-1377; Afzal M R, Samanta A, Shah Z I, Jeevanantham V, Abdel-Latif A, Zuba-Surma E K, Dawn B. Adult Bone Marrow Cell Therapy for Ischemic Heart Disease: Evidence and Insights From Randomized Controlled Trials. Circ Res 2015; 117(6):558-575). However, clinical studies have shown that mesenchymal stem cells could improve cardiac function after infarction to a certain extent through paracrine protection, although this effect is hardly noticable. Further studies have shown that the primary mechanism underlying the paracrine protection is by secretion of an extracellular vesicles, i.e., exosomes (Makridakis M, Roubelakis M G, Vlahou A. Stem cells: insights into the secretome. Biochim Biophys Acta 2013; 1834(11):2380-2384; Huang P, Tian X, Li Q, Yang Y. New strategies for improving stem cell therapy in ischemic heart disease. Heart Fail Rev 2016; 21(6):737-752; Lai R C, Arslan F, Lee M M, Sze N S, Choo A, Chen T S, Salto-Tellez M, Timmers L, Lee C N, El O R and others. Exosome secreted by MSC reduces myocardial ischemia/reperfusion injury. Stem Cell Res 2010; 4(3):214-222). Exosomes is advantageous in its rich sources and stability, with no immunogenicity, and BM-MSC-derived exosomes (MSC-Exo) can improve the myocardial infarction microenvironment. Thus, it is a promising next-generation myocardial repair product (Lamichhane T N, Sokic S, Schardt J S, Raiker R S, Lin J W, Jay S M. Emerging Roles for Extracellular Vesicles in Tissue Engineering and Regenerative Medicine. Tissue Engineering Part B: Reviews 2015; 21(1):45-54).

### SUMMARY OF THE INVENTION

An object of the present invention is to provide a preparation method of mesenchymal stem cell-derived exosomes with cardioprotective ability.

According to the study in the present invention, it has been discovered that pretreatment of mesenchymal stem cells with statins such as atorvastatin (ATV) can significantly improve the anti-apoptosis ability and homing ability of mesenchymal stem cells, and produce stem cell-derived exosomes with abilities of significantly improving myocardial infarction microenvironment and highly efficient myocardial repairing.

In one aspect, the present invention provides a preparation method of mesenchymal stem cell-derived exosomes, which method includes: pretreating mesenchymal stem cells with statins, and culturing the treated mesenchymal stem cells to collect exosomes secreted thereby.

According to a specific embodiment of the present invention, the preparation method of mesenchymal stem cell-derived exosomes of the invention includes:

adding statins into a medium of the mesenchymal stem cells for pretreatment for 12-24 hours, and then replacing the cell culture medium with a complete medium without exosome for continued culturing; after 48 hours, collecting the conditioned medium and isolating and obtaining exosomes secreted by the mesenchymal stem cells pretreated with statins by ultracentrifugation.

According to a specific embodiment of the present invention, in the preparation method of mesenchymal stem cell-derived exosomes of the invention, the ultracentrifugation process includes the steps of:

after collecting the conditioned medium, successively removing cells by centrifugation, cell debris by centrifugation, and large vesicles by high speed centrifugation, and then collecting the pellet by ultracentrifugation and re-suspending, and conducting further ultracentrifugation to obtain the exosome.

In one preferred specific embodiment of the present invention, the ultracentrifugation process includes steps of: after collecting the conditioned medium, removing cells by centrifugation at 300 g for 10 minutes, removing cell debris by centrifugation at 2,000 g for 20 minutes; removing large vesicles by high speed centrifugation at 16,500 g for 30 minutes; collecting the pellet by ultracentrifugation at 120,000 g for 70 minutes and re-suspending, and conducting further ultracentrifugation at 120,000 g for 70 minutes to obtain the exosomes.

According to a specific embodiment of the present invention, in the preparation method of mesenchymal stem cell-derived exosomes of the invention, the statins include atorvastatin.

According to a specific embodiment of the present invention, in the preparation method of mesenchymal stem cell-derived exosomes of the invention, the mesenchymal stem cells include bone marrow mesenchymal stem cells or adipose mesenchymal stem cells.

In another aspect, the present invention also provides exosomes prepared by the preparation method as described in the invention.

In still another aspect, the present invention also provides use of statins in the preparation of a formulation that promote the anti-apoptotic and/or homing ability of mesenchymal stem cells.

In yet another aspect, the present invention also provides use of statins in the preparation of a formulation that promote the secretion of exosomes having abilities of improving the myocardial infarction microenvironment and/or myocardial repairing from mesenchymal stem cells.

According to a specific embodiment of the present invention, in the invention, the statins include atorvastatin; preferably, mesenchymal stem cells were pretreated with 1 μM statins for 24 hours.

According to a specific embodiment of the present invention, in the invention, the mesenchymal stem cells include bone marrow mesenchymal stem cells or adipose mesenchymal stem cells.

In one specific embodiment of the present invention, exosomes capable effective myocardial repairing and endothelial protection can be obtained by pretreatment of BM-MSC with 1 μM ATV for 24 hours.

### BRIEF DESCRIPTION OF THE DRAWINGS

FIGS. 1A-1C show the identification results of the mesenchymal stem cell-derived exosome in an example of the present invention.

FIGS. 2A-2D show the difference in the effect of exosomes derived from mesenchymal stem cells pretreated with ATV at different concentrations on endothelial cells.

FIGS. 3A-3H show the test results of tube formation, migration and survival of vascular endothelial cells promoted by exosomes derived from ATV pretreated mesenchymal stem cells.

FIGS. 4A-4F show the test results in which exosomes derived from mesenchymal stem cells pretreated with ATV significantly improve cardiac function and reduce myocardial infarction area after myocardial infarction in rats.

FIGS. 5A-5K show the test results in which the protective effect of exosome derived from mesenchymal stem cell pretreated with ATV is related to its up-regulation of IncRNA H19.

### DETAILED DESCRIPTION

The present invention will be further explained with respect to its features and technical effects by means of the specific examples below, but the invention is not limited thereto in any way. In the Examples, all raw reagents and materials are commercially available. Experimental procedures with unspecified conditions are conventional procedures and conventional conditions well known in the related art, or in accordance with the conditions recommended by the instrument manufacturer.

### Example 1

Preparation of mesenchymal stem cell-derived exosomes: BM-MSCs of rats (Sprague-Dawley rats, 60-80 g) was isolated by differential adhesion and amplified via passage to the third-fourth generation for use. After 24 hours of pretreatment with ATV in BM-MSC culture medium (IMDM with 10% FBS and penicillin (100 U/mL)/streptomycin (100 mg/mL), the cell culture medium was replaced with exosome free FBS containing medium (IMDM medium containing 10% FBS after 18 hours of ultracentrifugation) for continued culturing. After 48 hours, the conditioned medium was collected and exosomes secreted by BM-MSCs pretreated with ATV (MSC^{ATV}-Exo) were isolated and obtained by ultracentrifugation. Specifically, the ultracentrifugation process included the steps of: after collecting the conditioned medium, removing cells by centrifugation at 300 g for 10 minutes, and removing cell debris by centrifugation at 2,000 g for 20 minutes; removing macrovesicles by high speed centrifugation at 16,500 g for 30 minutes; collecting the pellet by ultracentrifugation at 120,000 g for 70 minutes and re-suspending, and conducting further ultracentrifugation at 120,000 g for 70 minutes to obtain the exosome.

Identification of the prepared MSC^{ATV}-Exo: including electron microscopy (HITACHI, H-6001V, Japan) analysis of morphology and structures, NTA (Malvern Instruments, NanoSight, UK) analysis of particle size distribution of exosomes, and Western Blot assaying of exosome protein markers.

The effects of pretreatment with ATV at different concentrations on MSC^{ATV}-Exo function were compared, and the optimum ATV concentration for the pretreatment was selected. Functionality evaluation of MSC^{ATV}-Exo pretreated at this optimal ATV concentration was carried out, including the impact on tube formation, migration and anti-apoptosis of vascular endothelial cells, and the effect in improving cardiac function and reducing myocardial infarction area after myocardial infarction in rats upon intramyocardial injection. Finally, molecular biological evaluation, i.e., assay of the IncRNA H19 expression level in MSC^{ATV}-Exo, was conducted.

Evaluation Indicators (Research Results)

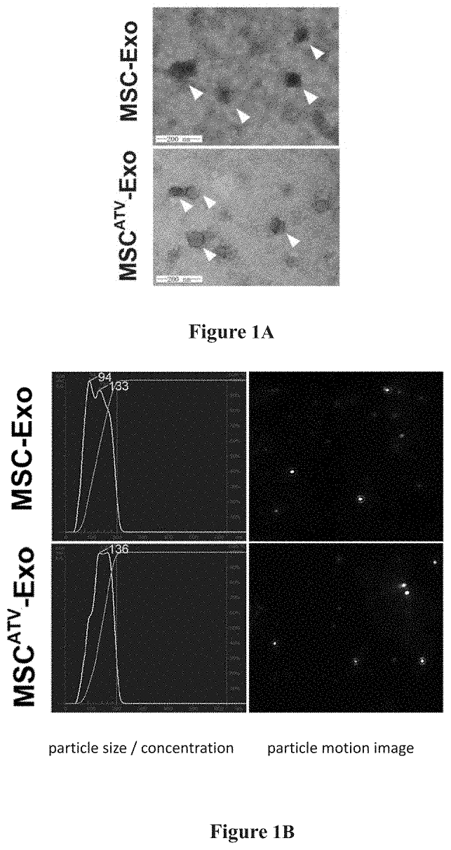

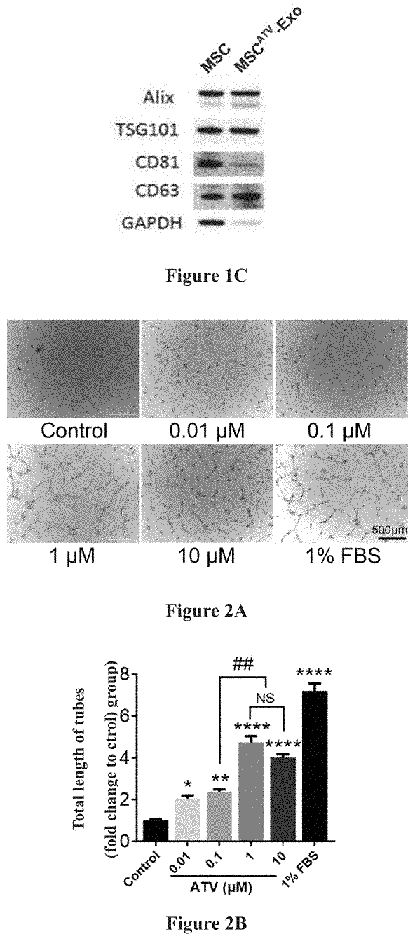

The MSC^{ATV}-Exo obtained by ultracentrifugation is spherical or disc shaped under the electron microscope, having a size of about 100 nm. NTA analysis shows a particle size distribution in the range of 30-150 nm. Western Blot assay shows high expression of exosome protein markers such as TSG101, Alix, CD63, and CD81 in MSC^{ATV}-Exo. No significant difference is evident in morphology, particle size distribution, and protein markers between the exosomes secreted by BM-MSCs pretreated with ATV or without pretreatment. Detailed results are demonstrated in FIG. 1A to FIG. 1C, and in FIG. 1A: the morphological structure of mesenchymal stem cell-derived exosomes (MSC-Exo) in a spherical or disc shape with a size of about 100 nm are observed under electron microscope, which is not changed after statins pretreatment; in FIG. 1B: the particle size distribution of MSC-Exo was analyzed by NTA, where both the MSC-Exo pretreated with statins and those without any pretreatment have a particle size distribution in the range of 30-150 nm; and in FIG. 1C: exosome protein markers are identified, where exosome protein markers such as TSG101, Alix, CD63, and CD81 were highly expressed in MSC-Exo pretreated with statins.

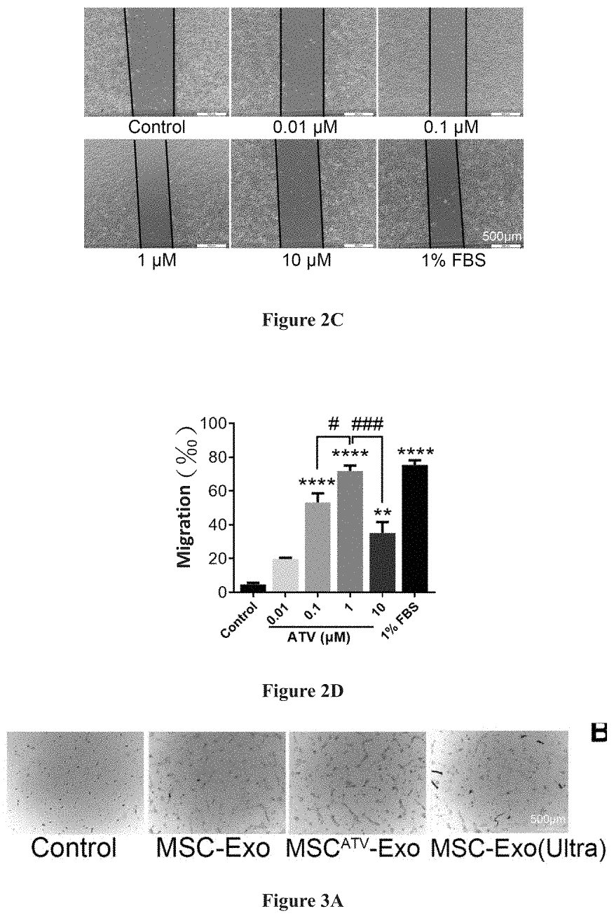

MSC^{ATV}-Exo obtained by pretreatment with ATV at different concentrations (0.01, 0.1, 1, 10 μM) were subjected to functionality analysis, in which it was found that MSC^{ATV}-Exo pretreated with 1 μM ATV had the most prominent effect on promoting tube formation and migration of endothelial cells. Detailed results are demonstrated in FIG. 2A to FIG. 2D, and in FIG. 2A to FIG. 2B: the effects of exosomes extracted from mesenchymal stem cells pretreated with ATV at different concentrations (0.01, 0.1, 1, 10 μM) on tube formation are compared, among which the 1 μM ATV pretreatment had the best effect (FIG. 2B); in FIG. 2C to FIG. 2D: the effects of exosomes extracted from mesenchymal stem cells pretreated with ATV at different concentrations on tube formation were compared, among which the 1 μM ATV pretreatment had the best effect (FIG. 2D).

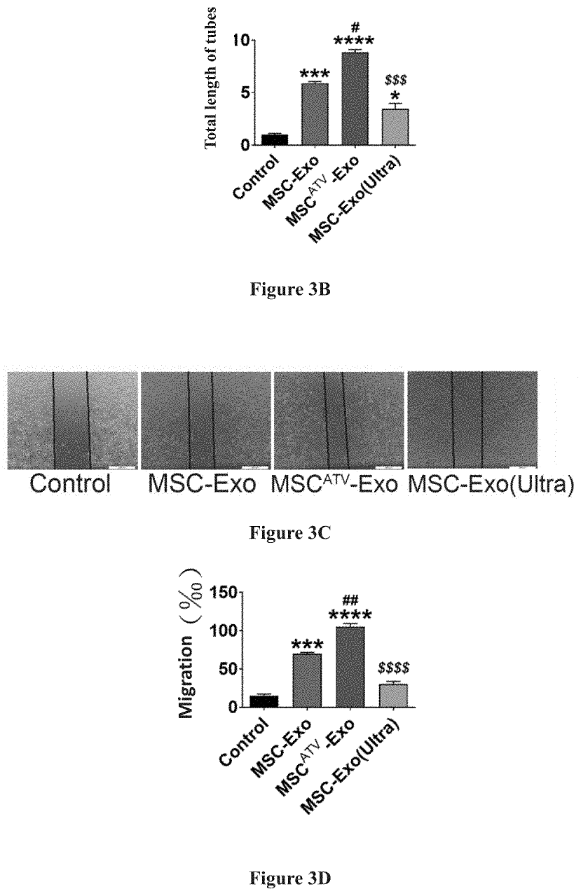

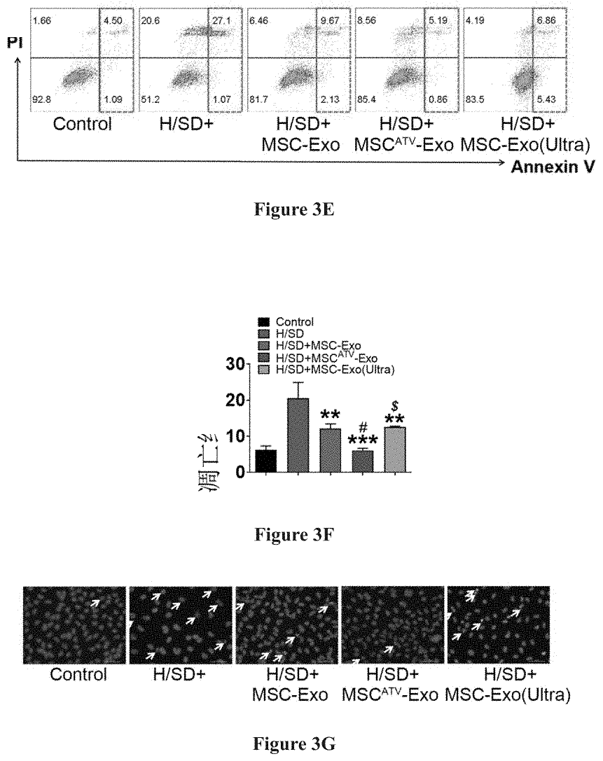

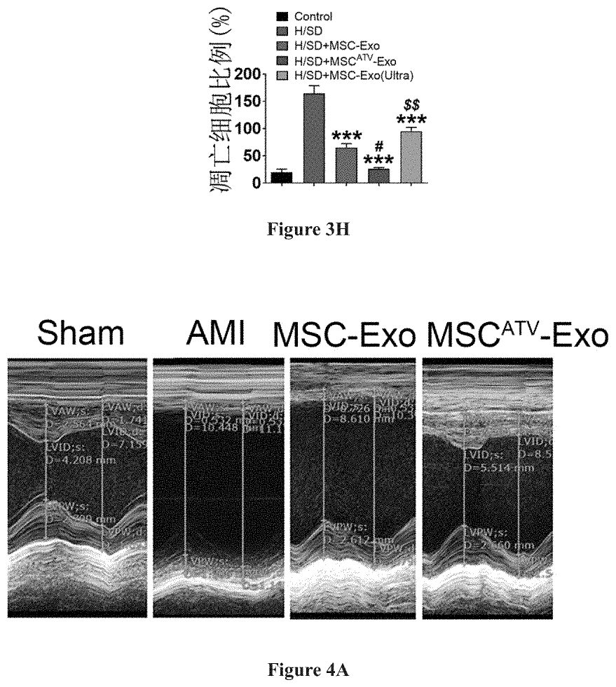

As compared to MSC-Exo without ATV pretreatment, MSC^{ATV}-Exo can significantly promote the tube formation and migration of endothelial cells, and promote the survival and anti-apoptosis of endothelial cells under hypoxia and serum deprived conditions. Detailed results are demonstrated in FIG. 3A to FIG. 3H, and FIG. 3A to FIG. 3B: in tube formation tests, the exosome derived from mesenchymal stem cells pretreated with ATV (MSC^{ATV}-Exo) significantly promotes tube formation of endothelial cells; in FIG. 3C to FIG. 3D: in migration tests, MSC^{ATV}-Exo significantly promotes the migration of endothelial cells as compared to the control group; in FIG. 3E to FIG. 3F: in flow cytometry assay, MSC^{A}TV-Exo significantly promotes the survival of endothelial cells under hypoxia and serum deprived conditions as compared to the control group; and in FIG. 3G to FIG. 3H: with Hoechst 33342 staining, MSC^{ATV}-Exo significantly reduces apoptosis of endothelial cells under hypoxia and serum deprived conditions as compared to the control group.

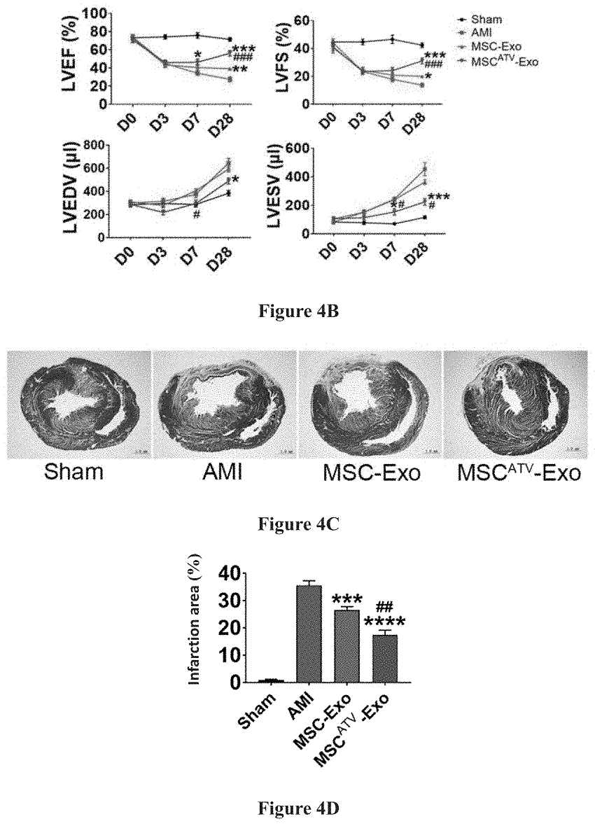

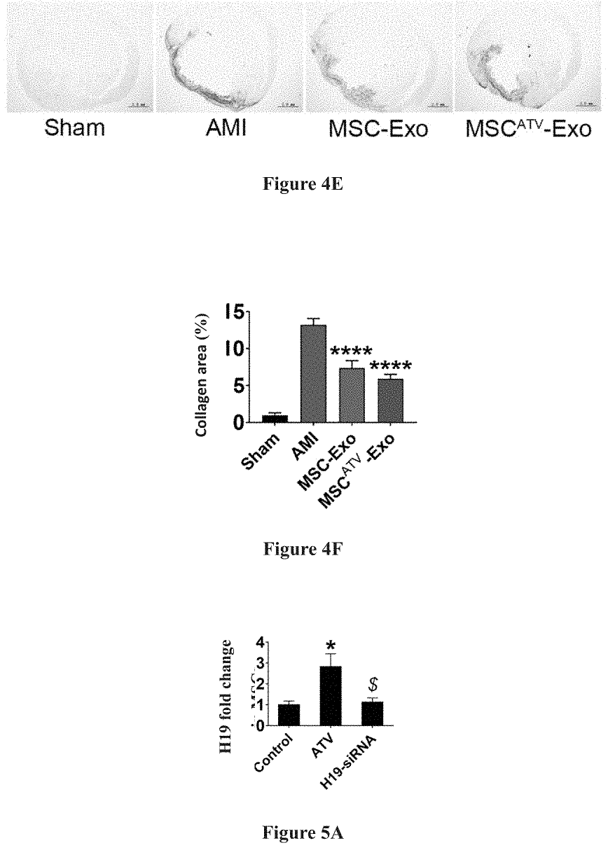

As compared to MSC-Exo without ATV pretreatment, upon its intramyocardial injection, MSC^{ATV}-Exo can significantly improve cardiac function and reduce myocardial infarction area after myocardial infarction in rats. Detailed results are demonstrated in FIG. 4A to FIG. 4F, and in FIG. 4A to FIG. 4B: transplantation of exosomes derived from mesenchymal stem cells pretreated with ATV (MSC^{ATV}-Exo) significantly improves the cardiac function in rats after myocardial infarction; in FIG. 4C to FIG. 4D: Masson staining shows that transplantation of MSC^{ATV}-Exo significantly reduces myocardial infarction area in rats; and FIG. 4E to FIG. 4F: Sirius red staining suggests that MSC^{ATV}-Exo transplantation remarkably reduce the local collagen deposition in rats after myocardial infarction.

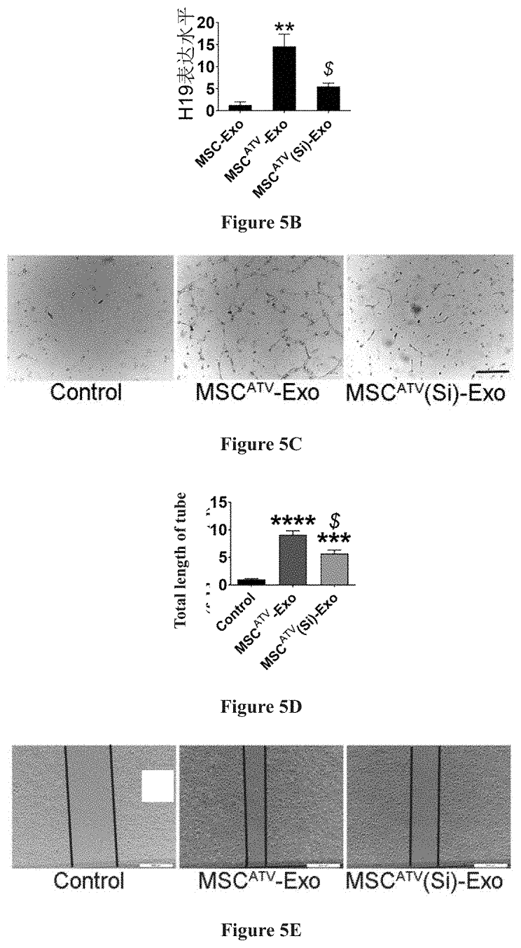

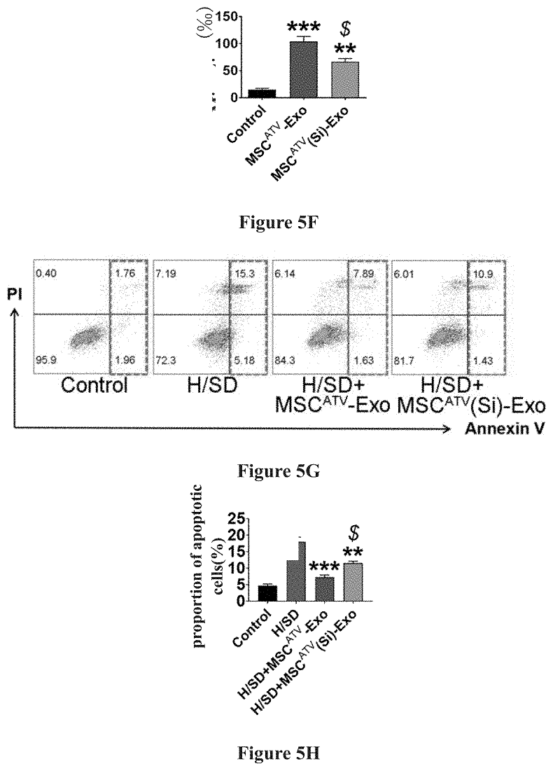

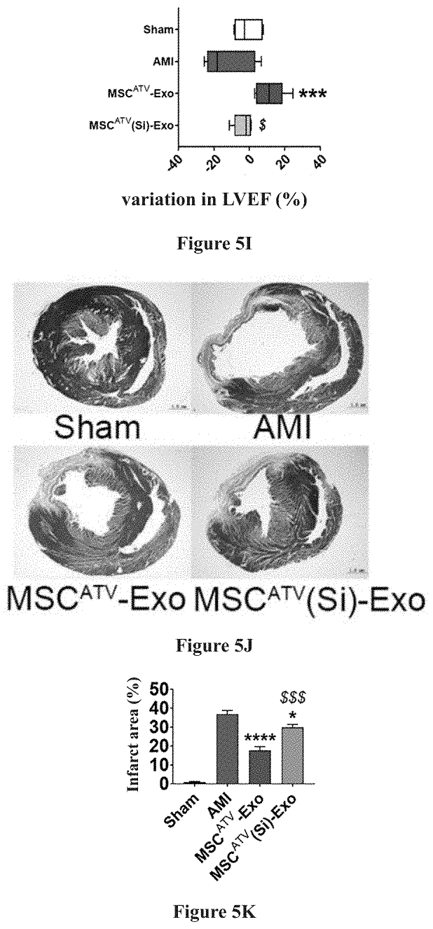

As compared to MSC-Exo, MSC^{A}TV-Exo highly expresses IncRNA H19 by up to 10 times or more. after the expression level of IncRNA H19 in MSC pretreated with ATV was knocked down by small interfering RNA, the exosome secreted thereby (MSC^{ATV}(Si)-Exo) was extracted, with the above discussed protective effects eliminated, suggesting that IncRNA H19 was related to the efficacy of MSC^{ATV}-Exo in endothelial cell protection, cardiac function improvement, and myocardial infarction area reduction. Detailed results are demonstrated in FIG. 5A to FIG. 5K, and in FIG. 5A to FIG. 5B: ATV pretreated mesenchymal stem cell-derived exosome (MSC^{ATV}-Exo) highly expresses IncRNA H19, whereas the expression of IncRNA H19 significantly decreased in the exosome with small interfering RNA knockdown (MSC^{ATV}(Si)-Exo); in FIG. 5C to FIG. 5H: in comparison to MSC^{ATV}-Exo, MSC^{A}TV(Si)-Exo has a lesser endothelial protective effect; and FIG. 5I to FIG. 5K: in comparison to MSC^{ATV}-Exo, MSC^{A}TV(Si)-Exo has lesser effects in improving cardiac function and repairing myocardium after infarction.

### CONCLUSION

Exosomes capable of effective endothelial protection and myocardial repairing can be obtained by pretreatment of BM-MSC with 1 μM ATV for 24 hours, and the mechanism thereof is related to the up-regulation of IncRNA H19 in the exosomes.

1. A preparation method of mesenchymal stem cell-derived exosomes including: pretreating mesenchymal stem cells with statins, and culturing the treated mesenchymal stem cells to collect exosomes secreted therefrom.

2. The method according to claim 1, including:
adding statins into a medium of the mesenchymal stem cells for pretreatment for 12-24 hours, and then replacing the cell culture medium with a exosome-free medium for continued culturing;
after 48 hours, collecting the conditioned medium and isolating exosomes derived from the mesenchymal stem cells pretreated with statins by repeated ultracentrifugation.

3. The method according to claim 2, wherein the ultracentrifugation process includes the steps of:
after collecting the conditioned medium, removing cells by centrifugation at 300 g for 10 minutes, removing cell debris by centrifugation at 2,000 g for 20 minutes; removing large vesicles by high speed centrifugation at 16,500 g for 30 minutes; collecting the pellet by ultracentrifugation at 120,000 g for 70 minutes and re-suspending, and conducting further ultracentrifugation at 120,000 g for 70 minutes to obtain the exosome.

4. The method according to claim 1, wherein the statins include atorvastatin.

5. The method according to claim 1, wherein the mesenchymal stem cells include bone marrow mesenchymal stem cells or adipose mesenchymal stem cells.

6. Exosomes prepared by the preparation method according to claim 1.

7. The exosomes according to claim 6, exhibiting an increased level of IncRNA H19 expression.

8. Use of statins in the preparation of a formulation that promote the secretion of exosomes from mesenchymal stem cells.

9. The use according to claim 8, wherein the exosomes have effects of accelerating migration, tube-like structure formation of endothelial cells, and increasing survival of endothelial cells under hypoxia and serum deprivation condition.

10. The use according to claim 8, wherein the exosomes have abilities of improving the myocardial infarction microenvironment and/or myocardial injury repair.

11. The use according to claim 8, wherein the statins include atorvastatin.

12. The use according to claim 11, wherein the mesenchymal stem cells were pretreated with 1 μM statins for 24 hours.

13. The use according to claim 8, wherein the mesenchymal stem cells include bone marrow mesenchymal stem cells or adipose mesenchymal stem cells.
